(19) United States
(12) Patent Application Publication
(10) Pub. No.: US 2021/0398077 A1
(43) Pub. Date: Dec. 23, 2021

(54) METHODS AND SYSTEMS FOR LEVERAGING HEALTHCARE CLAIMS FOR A HEALTHCARE PROVIDER SEARCH

(71) Applicant: HEALTHSPARQ, INC., Portland, OR (US)

(72) Inventors: Keith LoMurray, Salt Lake City, UT (US); Amanda Williams, Seattle, WA (US); Matt Jones, Poulsbo, WA (US)

(21) Appl. No.: 17/340,419

(22) Filed: Jun. 7, 2021

Related U.S. Application Data
(60) Provisional application No. 63/036,369, filed on Jun. 8, 2020.

(51) Int. Cl. G06Q 10/10; G16H 10/60; G06F 16/9538; G06F 16/9535

(52) U.S. Cl. CPC G06Q 10/10; G06F 16/9535; G06F 16/9538; G16H 10/60

(57) ABSTRACT

Systems and methods for searching for a healthcare provider based on an automatically-identified search context are provided. In one embodiment, a method comprises processing raw medical claims to identify an area of focus for a provider, associating the provider with the area of focus, receiving, from a user via a user device, a search query indicating a desired area of focus, and outputting, to the user device for display to the user, provider information of the provider if the desired area of focus comprises the area of focus. In this way, an area of focus of a provider may be automatically determined from raw claims data, and such an automatically-determined area of focus may be used to refine search results for users.

### CROSS-REFERENCE TO RELATED APPLICATIONS

This application claims priority from U.S. Provisional Patent Application No. 63/036,369, filed on Jun. 12, 2020, entitled, “Methods and Systems for Leveraging Healthcare Claims for a Healthcare Provider Search”, which is hereby incorporated by reference.

### BACKGROUND AND SUMMARY

Users seeking medical care or treatment may wish to select a particular physician or healthcare provider to visit to receive the care or treatment. Factors that may be considered when trying to select a particular provider may include costs, location, specialty, quality ratings, and so on. To that end, users may use a web-based search engine to search a directory of healthcare providers. However, users may not know what search terms to use and therefore may have difficulty finding appropriate providers. For example, a user with foot pain may simply search “foot doctor” instead of “podiatrist,” and a typical search engine will likely return a list of providers with names similar to “foot” but specialize in a field other than podiatry.

Improvements to such search engines for healthcare providers enable search results that more appropriately return “podiatrists” in response to a search query for “foot doctor,” for example by evaluating the search context and using structured dictionaries to refine the search. However, knowing a provider's specialty is not always enough to guide a patient to the right doctor because patients do not always know what specialty treats their specific healthcare need. Further, many providers focus on only a narrow range of care within their specialization. For example, an orthopedic surgeon may be licensed to perform numerous types of orthopedic surgeries, but specializes in a narrow range of surgeries, such as spinal surgeries, while not often or ever performing knee or hip replacements.

Previous care guidance solutions attempt to direct patients to an appropriate provider specializing in the needs of patients. For example, one common approach includes having the patient select a specialty and then providing a list of all providers within that specialty. The patient or a care navigator then contacts providers to determine which providers perform the needed care. In order to handle the problem that providers do not necessarily treat every specific condition within the purview of their specialty, some previous solutions have sought sub-specialization data on providers. However, such data is typically obtained by conducting surveys of healthcare providers. Obtaining such self-reported data is expensive to capture, may become quickly out-of-date, and is commonly inaccurate.

The inventors have recognized the above issues and have devised several approaches to address them. In particular, systems and methods for automatically identifying an area of focus of a healthcare provider are provided. In one embodiment, a method comprises processing raw medical claims to identify an area of focus for a provider, associating the provider with the area of focus, receiving, from a user via a user device, a search query indicating a desired area of focus, and outputting, to the user device for display to the user, provider information of the provider if the desired area of focus comprises the area of focus. In this way, an area of focus of a provider may be automatically determined from raw claims data, and such an automatically-determined area of focus may be used to refine search results for users.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE FIGURES

FIG. 1 illustrates an overview of an exemplary computing environment according to an embodiment.

FIG. 2 shows an overview of an exemplary arrangement of software modules for a search engine for finding healthcare providers according to an embodiment.

FIG. 3 shows a high-level flow chart illustrating an example method for leveraging healthcare claims to identify areas of focus of healthcare providers according to an embodiment.

FIG. 4 shows a high-level flow chart illustrating an example method for performing a search of healthcare providers based on areas of focus and a taxonomy according to an embodiment.

FIG. 5 shows a high-level flow chart illustrating an example method for processing healthcare claims to identify areas of focus for healthcare providers according to an embodiment.

FIG. 6 shows a high-level flow chart illustrating an example method for evaluating claims for an encounter to determine areas of focus and patient attributes associated with providers according to an embodiment.

FIG. 7 shows a high-level flow chart illustrating an example method for evaluating associations for providers to determine areas of focus for the providers according to an embodiment.

FIGS. 8A-8C show a set of tables of example claims illustrating a method for evaluating claims to identify areas of focus of providers according to an embodiment.

FIG. 9 shows a table illustrating example areas of focus associated with a provider according to an embodiment.

FIG. 10 shows a table illustrating example data for determining areas of focus for providers according to an embodiment.

FIGS. 11A-11B show a diagram illustrating an example graphical user interface for displaying search results based on areas of focus according to an embodiment.

### DETAILED DESCRIPTION

The present description relates to systems and methods of searching for a healthcare provider. Currently, when a user searches for a healthcare provider, searches for “hand surgeon” and “back surgeon” may return the same orthopedic surgeons, regardless of whether the orthopedic surgeons even perform hand or back surgery. As discussed hereinabove, data regarding areas of focus for providers may alleviate the issue of returning the same results, but capturing accurate and robust data regarding areas of focus is currently difficult, as self-reported data is notoriously inaccurate and expensive to capture. Thus, systems and methods for determining an area of focus of a healthcare provider and performing searches for healthcare providers based on determined areas of focus are provided.

As described herein, the term “area of focus” refers to medical specialties (e.g., Family Medicine, Emergency Medicine, Dermatology, Obstetrics and Gynecology, and so on), but may more specifically refers to subspecialties (e.g., abdominal radiology, addiction psychiatry, cardiovascular disease, craniofacial surgery, hand surgery, female pelvic medicine and reconstructive surgery, and so on), specific body parts that are treated (e.g., head, knee, shoulder, hand, spine, and so on), specific imaging technologies used (e.g., ultrasound, MRI, and so on), and other high-level groupings of medical procedures or healthcare services. For example, while orthopedic surgeons may be licensed to treat many different areas of the body, over half of all orthopedic surgeons are siloed into a single area of focus such that they treat only one or two specific body parts, while very few orthopedic surgeons regularly treat more than three or four areas of the body.

A healthcare claim or medical claim is a bill for healthcare services that healthcare providers provide to healthcare insurers for payment. Such medical claims are based on standardized medical codes (e.g., a current procedural terminology (CPT) code), wherein a procedure or diagnosis is indicated via a procedure code or diagnosis code, rather than in natural language as the claims are intended for use in insurance processing than for a review of a medical history of the patient. In addition to the standardized medical codes, medical claims may include additional information regarding the cost of services. In contrast, a medical record may include clinical data and medical history for a patient, such as demographics, vital signs, diagnoses, medications, treatment plans, progress notes, problems, immunization dates, allergies, radiology images, laboratory results, and test results. Thus, as referred to herein, the term “raw medical claims,” also referred to interchangeably as “healthcare claims” and “medical claims,” refers to code-based health insurance claims rather than medical records data. The raw medical claims data may comprise health insurance claims data from health insurers, for example, and/or insurance claims data aggregated from multiple payer datasets such as an all-payer claims dataset. As described further herein, the systems and methods provided in the present disclosure leverage medical claims data to identify areas of focus for healthcare providers, as medical claims directly link healthcare providers to medical procedures and diagnoses that they provide.

To leverage healthcare claims to identify areas of focus, the systems provided herein develop taxonomies that map procedures performed by healthcare providers to specific areas of focus. For example, particular procedures may be mapped to areas of the body in a taxonomy, which may also enable natural language description of such procedures which are typically encoded in claims as procedure codes. Thus, taxonomies for areas of focus may indicate specific body parts treated within a subspecialty. Other taxonomies may indicate durable medical equipment, medical imaging services, various types of physical therapy including speech and occupational therapy, allergy providers, gastroenterologists who perform biopsies, and so on. Taxonomies may also be provided to designate areas of focus for behavioral health.

Further, claims data may be evaluated with respect to patients to identify certain patient attributes, and these identified patient attributes may be considered when evaluating the procedures performed by a given healthcare provider in order to identify providers who serve a higher percentage of patients with those patient attributes. As one illustrative and non-limiting example, it can be difficult for transgender patients to find providers with the experience to focus on their unique needs. Most transgender patients rely on word-of-mouth or self-made directories of providers to find transgender-friendly healthcare providers. Using the systems and methods provided herein, claims data may be leveraged to identify which providers treat a greater-than-average number of transgender patients. Users may then identify their reason for care, which may be seemingly unrelated to specific transgender issues, but also indicate that they are seeking a transgender-friendly provider, and the systems provided herein may provide search results including providers that focus on transgender patients. Other categories for patient attributes may include age (e.g., use patient age to find providers who focus on adolescent or geriatric populations), condition specific (e.g., use diagnosis codes to help find a provider that cares for people with a specific condition, such as an obstetrician who specializes in diabetic patients), and for cultural competency (e.g., providers who are LGBTQ+ friendly or who specialize in treating patients with autism).

The systems and methods described herein provide a number of benefits. Users are less overwhelmed by a large volume of marginally-relevant search results. Users may be directed to healthcare providers who specialize in a desired treatment or area, rather than providers who may be licensed in a given specialty but focus on other treatments or areas. Further, users may search for providers with a proven cultural competency for treating patients within a given patient population. Further still, the process may be updated on a regular basis and the specialization of a provider within a category may be weighted based on their history of performing in that area of focus. In this way, providers for whom an area of focus changes over time may be identified with the more recent area of focus. Further, providers with a long history and recent evidence would score high, thereby informing users of the provider's high performance in the area.

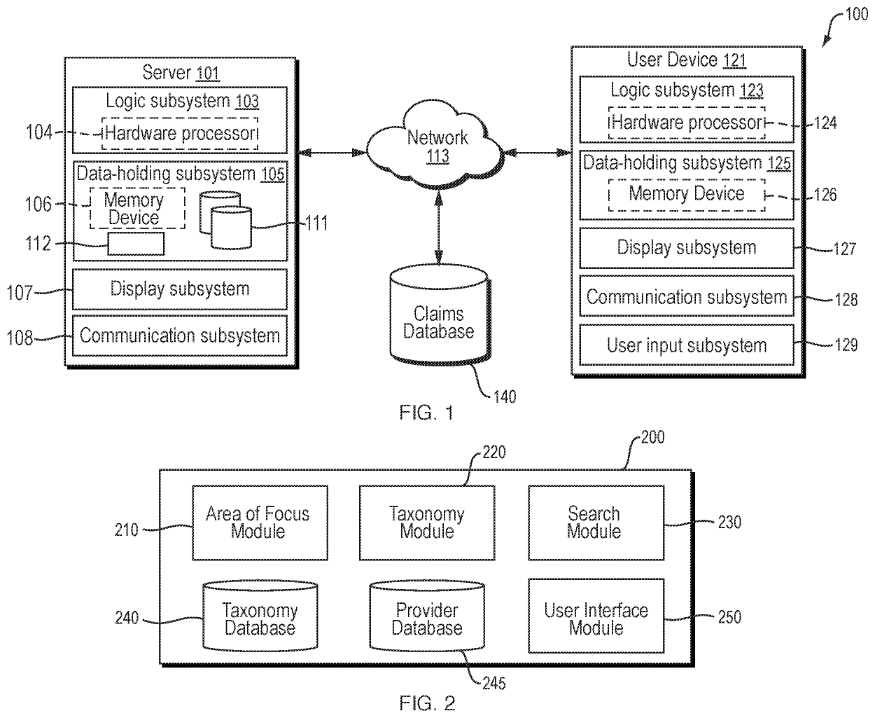

FIG. 1 illustrates an example computing environment 100 in accordance with the present disclosure. In particular, computing environment 100 includes a server 101, a plurality of user devices or client systems 121, and a network 113. However, not all of the components illustrated may be required to practice the invention. Variations in the arrangement and type of the components may be made without departing from the spirit or scope of the invention.

Server 101 may be a computing device configured to determine search contexts based on query input to narrow a search for a healthcare provider. In different embodiments, server 101 may take the form of a mainframe computer, server computer, desktop computer, laptop computer, tablet computer, home entertainment computer, network computing device, mobile computing device, mobile communication device, gaming device, and so on.

Server 101 includes a logic subsystem 103 and a data-holding subsystem 105. Server 101 may optionally include a display subsystem 107, communication subsystem 108, and/or other components not shown in FIG. 1. For example, server 101 may also optionally include user input devices such as keyboards, mice, game controllers, cameras, microphones, and/or touch screens.

Logic subsystem 103 may include one or more physical devices configured to execute one or more instructions. For example, logic subsystem 103 may be configured to execute one or more instructions that are part of one or more applications, services, programs, routines, libraries, objects, components, data structures, or other logical constructs. Such instructions may be implemented to perform a task, implement a data type, transform the state of one or more devices, or otherwise arrive at a desired result.

Logic subsystem 103 may include one or more hardware processors 104 that are configured to execute software instructions. Additionally or alternatively, the logic subsystem 103 may include one or more hardware or firmware logic machines configured to execute hardware or firmware instructions. Processors of the logic subsystem 103 may be single or multi-core, and the programs executed thereon may be configured for parallel or distributed processing. The logic subsystem 103 may optionally include individual components that are distributed throughout two or more devices, which may be remotely located and/or configured for coordinated processing. One or more aspects of the logic subsystem 103 may be virtualized and executed by remotely accessible networked computing devices configured in a cloud computing configuration.

Data-holding subsystem 105 may include one or more physical, non-transitory memory devices 106 configured to hold data and/or instructions executable by the logic subsystem 103 to implement the herein described methods and processes. When such methods and processes are implemented, the state of data-holding subsystem 105 may be transformed (for example, to hold different data).

Data-holding subsystem 105 may include removable media and/or built-in devices. Data-holding subsystem 105 may include optical memory (for example, CD, DVD, HD-DVD, Blu-Ray Disc, etc.), and/or magnetic memory devices (for example, hard drive disk, floppy disk drive, tape drive, MRAM, etc.), and the like. Data-holding subsystem 105 may include devices with one or more of the following characteristics: volatile, nonvolatile, dynamic, static, read/write, read-only, random access, sequential access, location addressable, file addressable, and content addressable. In some embodiments, logic subsystem 103 and data-holding subsystem 105 may be integrated into one or more common devices, such as an application-specific integrated circuit or a system on a chip.

It is to be appreciated that data-holding subsystem 105 includes one or more physical, non-transitory devices. In contrast, in some embodiments aspects of the instructions described herein may be propagated in a transitory fashion by a pure signal (for example, an electromagnetic signal) that is not held by a physical device for at least a finite duration. Furthermore, data and/or other forms of information pertaining to the present disclosure may be propagated by a pure signal.

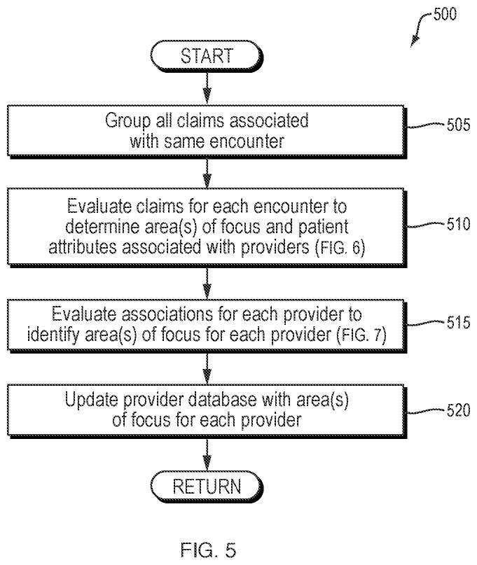

As discussed further herein, the data-holding subsystem 105 stores a system for processing medical claims and performing searches of healthcare providers in the form of executable instructions 112. When the executable instructions 112 are executed by one or more hardware processors 104 of the logic subsystem 103 to run the search system on the one or more hardware processors 104 of the logic subsystem 103, the logic subsystem 103 is operable to process raw medical claims to automatically identify one or more areas of focus for healthcare providers. The raw medical claims may be retrieved from a claims database, such as a claims database 140 communicatively coupled to the server 101 via the network 113, as depicted. Alternatively, the data-holding subsystem 105 may store the claims database 140 as one of the plurality of databases 111. An example method for processing raw medical claims to automatically identify one or more areas of focus for healthcare providers is described further herein with regard to FIGS. 3 and 5-7.

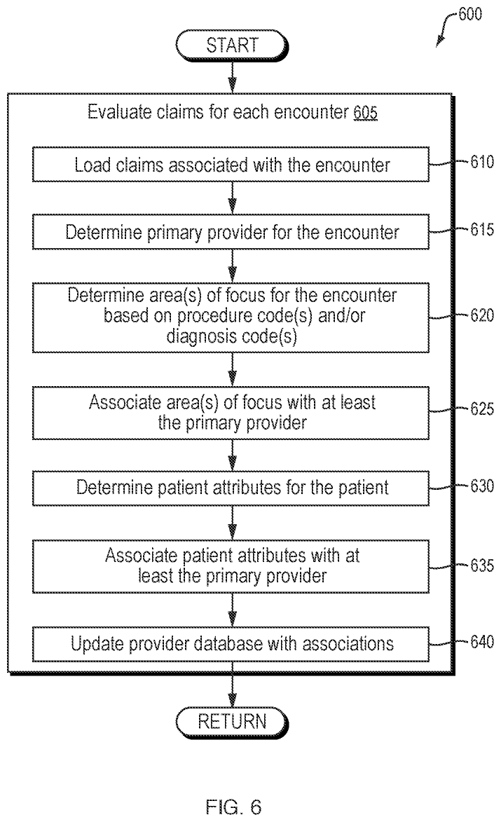

Additionally, when the executable instructions 112 are executed by one or more hardware processors 104 of the logic subsystem 103, the logic subsystem 103 is further operable to perform a search of one or more databases 111 including one or more provider databases. A provider database stores a list or a table of health care providers. The provider database may further include additional metadata for each provider, including but not limited to provider specialties, location information (e.g., address, business hours, accessibility), availability (e.g., whether or not the provider is accepting new patients), descriptive information (e.g., gender, one or more photographs, a biographical description, language(s) spoken), automatically-identified area(s) of focus, and so on. To search the provider database, the logic subsystem 103 may identify one or more desired areas of focus from a search query submitted by a user, and limit the search of the provider database to the one or more desired areas of focus. An example method for performing a search of healthcare providers according to an area of focus is described further herein with regard to FIG. 4.

When included, display subsystem 107 may be used to present a visual representation of data held by data-holding subsystem 105. As the herein described methods and processes change the data held by the data-holding subsystem 105, and thus transform the state of the data-holding subsystem 105, the state of display subsystem 107 may likewise be transformed to visually represent changes in the underlying data. Display subsystem 107 may include one or more display devices utilizing virtually any type of technology. Such display devices may be combined with logic subsystem 103 and/or data-holding subsystem 105 in a shared enclosure, or such display devices may be peripheral display devices.

When included, communication subsystem 108 may be configured to communicatively couple server 101 with one or more other computing devices, such as user device 121. Communication subsystem 108 may include wired and/or wireless communication devices compatible with one or more different communication protocols. As non-limiting examples, communication subsystem 108 may be configured for communication via a wireless telephone network, a wireless local area network, a wired local area network, a wireless wide area network, a wired wide area network, etc. In some embodiments, communication subsystem 108 may allow server 101 to send and/or receive messages to and/or from other devices via a network such as the public Internet. For example, communication subsystem 108 may communicatively couple server 101 with user device 121 via network 113. In some examples, network 113 may be the public Internet. In other examples, network 113 may be regarded as a private network connection and may include, for example, a virtual private network or an encryption or other security mechanism employed over the public Internet.

Further, the server 101 provides a network service that is accessible to a plurality of users through a plurality of client systems including the user device 121 communicatively coupled to the server 101 via a network 113. As such, computing environment 100 may include one or more devices operated by users, such as user device 121. User device 121 may be any computing device configured to access a network such as network 113, including but not limited to a personal computer, a laptop, a smartphone, a tablet, and the like. While one user device or client system 121 is shown, it should be appreciated that any number of user devices may be communicatively coupled to the server 101 via the network 113.

User device 121 includes a logic subsystem 123 and a data-holding subsystem 125. User device 121 may optionally include a display subsystem 127, communication subsystem 128, and/or other components not shown in FIG. 1. The user device 121 may be operated by a user such as a person searching for care for themselves (e.g., a patient) or a person searching for care on behalf of another person (e.g., a care navigator such as a health plan employee or concierge service). The user (such as the patient or care navigator, for example) may thus use the user device 121 to access, via the network 113, a network service provided by the server 101, wherein the network service enables the user to search for healthcare providers according to an area of focus as described herein. To that end, the user device 121 may display a graphical user interface, such as the graphical user interface described further herein with regard toFIGS. 11A-11B, via the display subsystem 127 to enable access for the user to the network service.

Logic subsystem 123 may include one or more physical devices configured to execute one or more instructions. For example, logic subsystem 123 may be configured to execute one or more instructions that are part of one or more applications, services, programs, routines, libraries, objects, components, data structures, or other logical constructs. Such instructions may be implemented to perform a task, implement a data type, transform the state of one or more devices, or otherwise arrive at a desired result.

Logic subsystem 123 may include one or more hardware processors 124 that are configured to execute software instructions. Additionally or alternatively, the logic subsystem 123 may include one or more hardware or firmware logic machines configured to execute hardware or firmware instructions. Processors of the logic subsystem 123 may be single or multi-core, and the programs executed thereon may be configured for parallel or distributed processing. The logic subsystem 123 may optionally include individual components that are distributed throughout two or more devices, which may be remotely located and/or configured for coordinated processing. One or more aspects of the logic subsystem 123 may be virtualized and executed by remotely accessible networking computing devices configured in a cloud computing configuration.

Data-holding subsystem 125 may include one or more physical, non-transitory memory devices 126 configured to hold data and/or instructions executable by the logic subsystem 123 to implement the herein described methods and processes. When such methods and processes are implemented, the state of data-holding subsystem 125 may be transformed (for example, to hold different data).

Data-holding subsystem 125 may include removable media and/or built-in devices. Data-holding subsystem 125 may include optical memory (for example, CD, DVD, HD-DVD, Blu-Ray Disc, etc.), and/or magnetic memory devices (for example, hard drive disk, floppy disk drive, tape drive, MRAM, etc.), and the like. Data-holding subsystem 125 may include devices with one or more of the following characteristics: volatile, nonvolatile, dynamic, static, read/write, read-only, random access, sequential access, location addressable, file addressable, and content addressable. In some embodiments, logic subsystem 123 and data-holding subsystem 125 may be integrated into one or more common devices, such as an application-specific integrated circuit or a system on a chip.

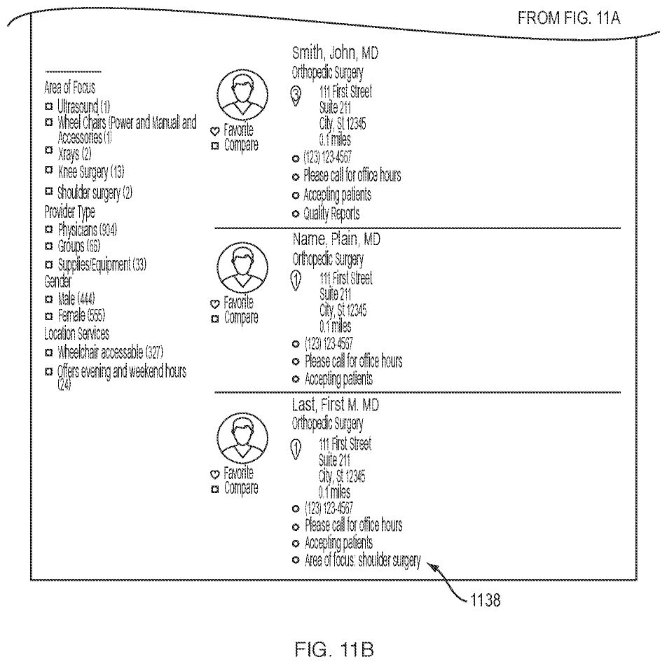

When included, display subsystem 127 may be used to present a visual representation of data held by data-holding subsystem 125. As the herein described methods and processes change the data held by the data-holding subsystem 125, and thus transform the state of the data-holding subsystem 125, the state of display subsystem 127 may likewise be transformed to visually represent changes in the underlying data. Display subsystem 127 may include one or more display devices utilizing virtually any type of technology. Such display devices may be combined with logic subsystem 123 and/or data-holding subsystem 125 in a shared enclosure, or such display devices may be peripheral display devices. The display subsystem 127 may display a graphical user interface for facilitating a search of healthcare providers. An example graphical user interface that may be displayed via the display subsystem 127 is described further herein with regard to FIGS. 11A-11B.

When included, communication subsystem 128 may be configured to communicatively couple user device 121 with one or more other computing devices, such as server 101. Communication subsystem 128 may include wired and/or wireless communication devices compatible with one or more different communication protocols. As non-limiting examples, communication subsystem 128 may be configured for communication via a wireless telephone network, a wireless local area network, a wired local area network, a wireless wide area network, a wired wide area network, etc. In some embodiments, communication subsystem 128 may allow user device 101 to send and/or receive messages to and/or from other devices, such as server 101, via a network 113 such as the public Internet.

User device 121 may further include a user input subsystem 129 comprising user input devices such as keyboards, mice, game controllers, cameras, microphones, and/or touch screens. A user of user device 121 may input a search query, for example, via user input subsystem 129. As discussed further herein, user device 121 may stream, via communication subsystem 128, user input received via the user input subsystem 129 to the server 101 in real time over the network 113. In this way, the server 101 may automatically suggest a search query based on the user input before the user formally submits a query to the server 101.

Thus server 101 and user device 121 may each represent computing devices which may generally include any device that is configured to perform computation and that is capable of sending and receiving data communications by way of one or more wired and/or wireless communication interfaces. Such devices may be configured to communicate using any of a variety of network protocols. For example, user device 121 may be configured to execute a browser application that employs HTTP to request information from server 101 and then displays the retrieved information to a user on a display. As mentioned hereinabove, an example graphical user interface that may be delivered to the user device 121 from the server 101 in such a manner and displayed, for example, on display subsystem 127 is described further herein and with regard to FIGS. 11A-11B.

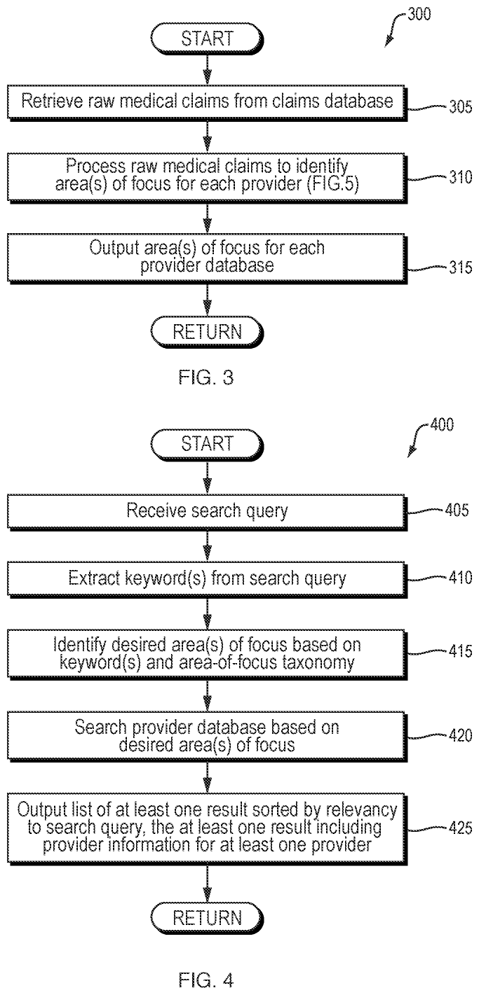

FIG. 2 shows an overview of an exemplary arrangement of software modules for a healthcare provider search system 200 for implementing a search engine for finding healthcare providers. In particular, the search system 200 may include an area-of-focus module 210, a taxonomy module 220, a search module 230, a taxonomy database 240, a provider database 245, and a user interface module 250. The search system 200 may include additional modules not shown, including but not limited to a suggest module for suggesting search terms based on a received search query, a spellcheck module for evaluating and correcting spelling of terms in a search query, a score module for scoring search results, and so on. The search system 200 may, for example, be included as executable instructions 112 stored in a non-transitory memory device 106 of the data-holding subsystem 105 of server 101.

The area-of-focus module 210 processes raw medical claims to identify one or more areas of focus associated with healthcare providers. For example, the area-of-focus module 210 analyzes claims data to identify providers based on similar procedures they perform, conditions they treat based on the diagnosis, and providers who specialize in seeing certain types of patients such as patients with autism or LGBTQ+ patients. The area-of-focus module 210 may process health insurance claims data from health insurers, for example, and/or data aggregated from multiple payer datasets.

The taxonomy module 220 provides a detailed taxonomy for each specific area of focus. As an illustrative example, the taxonomy module 220 may create a taxonomy for orthopedic surgery listing different areas of the body (e.g., hand, shoulder, back, hip, knee, and so on) and connects or groups these areas of the body to different medical procedures and/or diagnoses associated with orthopedic surgery. The taxonomy module 220 further connects the medical procedures and diagnoses, which may be expressed in the taxonomy through plain or human-readable text or labels, to corresponding diagnosis and/or procedure codes. As another example, the taxonomy module 220 may create a taxonomy for behavioral health that connects a taxonomy of behavioral disorders to diagnosis codes, such that the taxonomy may be used by the area-of-focus module 210 to identify which behavioral health providers treat different types of disorders. The taxonomies created and maintained by the taxonomy module 220 may be stored in a taxonomy database 240.

Taxonomies may be created by identifying all services or diagnoses performed within a specialty and curating a list of services or diagnoses within the specialty that are similar. The taxonomy module 220, in some examples, may automatically generate such taxonomies by clustering providers into bins using a machine learning clustering algorithm and then applying a label to the group.

As an example, a plurality of taxonomies may be linked to orthopedic surgery, where each taxonomy relates to a part of the body or an “area of focus.” For example, a taxonomy of procedures may link an “Arm” area of focus to procedures including, but not limited to, elbow tenotomy, arthrotomy of elbow, arthrotomy of wrist joint. As another example, a “Hand” area of focus may be linked to procedures including carpal tunnel, carpal tunnel with scope, palm-only fasciectomy, hand arthroplasty, and repair finger tendon. As yet another example, a taxonomy for a “Shoulder” area of focus may include procedures such as rotator cuff repair (surgical, non-arthroscopic), shoulder arthroscopy, and shoulder arthroscopy with rotator cuff repair. As another example, a taxonomy for a “Hip” area of focus may include procedures such as hip replacement. As another example, a taxonomy for a “Back” area of focus for orthopedic surgery may include procedures such as back surgery (laminectomy outpatient), combined anterior/posterior spinal fusion, disk surgery upper-back/neck, insertion or replacement of spinal neurostimulator pulse generator, insertion of spinal canal catheter, inpatient laminectomy, cervical spinal fusion, lumbar spinal fusion, spinal fusion of neck, and spinal injection for pain management. As another example, a taxonomy for a “Foot” area of focus may include procedures such as bunionectomy and hammertoe correction. As yet another example, a taxonomy for a “Knee” area of focus for orthopedic surgery may include procedures such as ACL repair by arthroscopy, knee arthroscopy with cartilage repair, knee replacement, and repair of patella. As yet another example, a taxonomy for a “Leg” area of focus for orthopedic surgery may include procedures such as bilateral or multiple major joint procedures of lower extremity, lower extremity and humerus procedure (except hip, foot, and femur), and revision of total hip or total knee replacement. As another example, a taxonomy for a “Tendon” area of focus for orthopedic surgery may include procedures such as steroid injection into tendon, and treatment of tendonitis.

Taxonomies may also be provided to designate areas of focus for behavioral health. For example, diagnosis codes may be used to define areas of focus for behavioral health. As an example, a “Mood Disorder” area of focus may include diagnosis codes for bipolar, major depression, and other mood disorders; a “Chemical Dependency” area of focus may include diagnosis codes for alcohol, cannabis, opioids, cocaine, stimulants, sedatives; an “Anxiety” area of focus may include diagnoses for generalized anxiety disorder, panic disorder, phobias, and other anxiety disorders; a “Neurodevelopmental” area of focus may include diagnoses for ADHD, autistic disorder, pervasive development disorders, and intellectual abilities; an “Eating Disorder” area of focus may include diagnoses for anorexia nervosa and bulimia nervosa; a “Suicide Ideation” area of focus may include diagnoses for suicide attempts and suicide ideation; and so on. Such taxonomies further enable the identification of providers who focus on particular diagnoses within an area of focus, which in turn enables users to search for providers who specifically treat such diagnoses.

The area-of-focus module 210 may thus leverage the taxonomies of the taxonomy module 220 and stored in the taxonomy database 240 to identify areas of focus for healthcare providers based on the types of procedures and diagnoses provided by the healthcare providers. Furthermore, the area-of-focus module 210 may also examine patient attributes of patients treated by each provider to identify whether the provider treats a substantially high percentage of patients with a given attribute. To that end, rather than relying on a taxonomy, the area-of-focus module 210 evaluates all procedures and/or diagnosis codes for a given patient to identify patient attributes that may not directly relate to other procedures or diagnoses. For example, certain diagnosis and/or procedure codes may indicate that a patient is a transgender patient, and the area-of-focus module 210 may identify such a patient attribute. The area-of-focus module 210 may then determine, when evaluating procedures and diagnoses provided by a given healthcare provider, that the provider treats a number of patients with a given patient attribute above a baseline rate relative to other healthcare providers, even if the procedure and/or diagnosis codes directly associated with the healthcare provider do not indicate the patient attribute. The provider may thus be associated with the patient attribute, which may be considered an area of focus. In this way, patients may search for healthcare providers who specialize in treating certain segments of the patient population.

The search module 230 drives the search API which is responsible for performing specific searches on specific provider attributes. The search module 230 receives a search query submitted by a user, evaluates the search query to identify keywords, and leverages the area-of-focus module 210 and/or the taxonomy module 220 to connect the keywords to specific areas of focus. The search module 230 then searches the provider database 245 based on the identified areas of focus. An example method for searching for healthcare providers according to a desired area of focus is described further herein with regard to FIG. 4.

The user interface module 250 generates and maintains user interfaces such as graphical user interfaces or textual user interfaces. The user interface module 250 further transmits data to user devices, such as user device 121, for generating the user interface at the user device 121. An example user interface is described further herein with regard to FIGS. 11A-11B.

FIG. 3 shows a high-level flow chart illustrating an example method 300 for leveraging healthcare claims to identify areas of focus of healthcare providers according to an embodiment. In particular, method 300 relates to automatically processing raw medical claims data to identify areas of focus for healthcare providers. Method 300 is described with reference to the systems and components of FIGS. 1 and 2, though it should be appreciated that the method may be implemented with other systems and components without departing from the scope of the present disclosure. Method 300 may be stored as executable instructions in non-transitory memory 106, for example, on server 101 and may be executed by one or more hardware processors 104 of the logic subsystem 103.

Method 300 begins at 305. At 305, method 300 retrieves raw medical claims from a claims database. The raw medical claims may include all claims for all services. The raw medical claims are designed for billing purposes and thus are not structured to indicate an area of focus of a provider. Instead, the raw medical claims are based on codes rather than natural language, and include information about who received care, who provided the care, a procedure or service performed, the diagnosis leading to care, the cost, the health insurance network, and so on. Method 300 may retrieve the raw medical claims from a remote claims database, such as claims database 140, or from a local claims database in the plurality of databases 111.

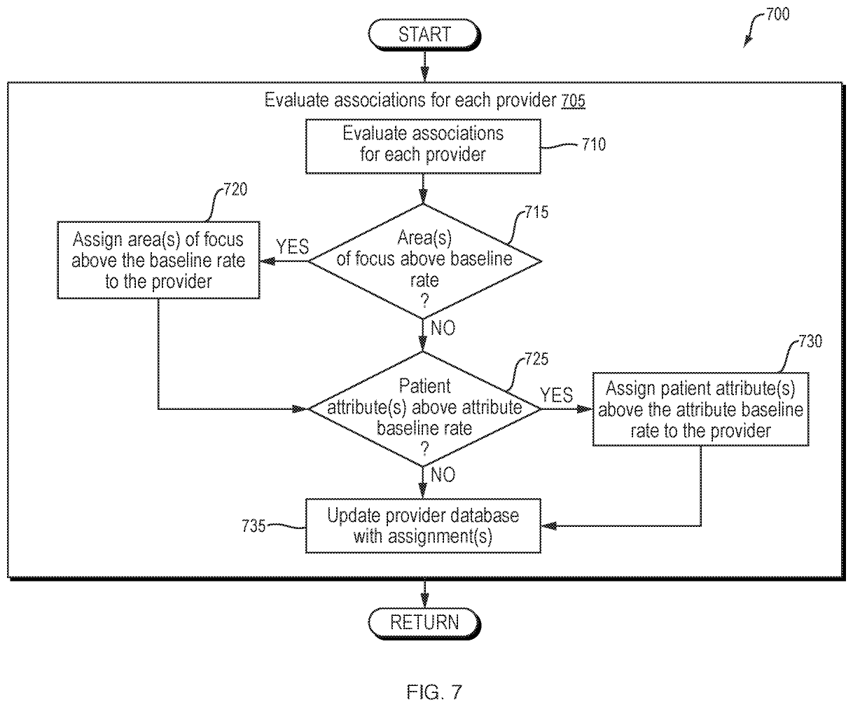

At 310, method 300 processes the raw medical claims to identify one or more areas of focus for each provider. An example method for processing the raw medical claims to identify area(s) of focus for each provider, such as the method described further herein with regard to FIG. 5, includes evaluating claims for each encounter to determine areas of focus and patient attributes associated with providers, and then evaluating associations for each provider to identify areas of focus for each provider. The area of focus assigned or designated to a provider may thus comprise a metric based on the amount of evidence that the provider treats or focuses on treating within that area of focus.

At 315, method 300 outputs the area(s) of focus for each provider determined from the raw medical claims to a provider database. For example, method 300 may output designations or assignments of an area of focus for each provider to a provider database 245. Method 300 then returns.

FIG. 4 shows a high-level flow chart illustrating an example method 400 for performing a search of healthcare providers based on areas of focus and a taxonomy according to an embodiment. In particular, method 400 relates to automatically identifying a desired area of focus based on query input, and performing a search for a healthcare provider based on the identified area of focus. In this way, the relevancy of the search results may be increased. Method 400 is described with reference to the systems and components of FIGS. 1 and 2, though it should be appreciated that the method may be implemented with other systems and components without departing from the scope of the present disclosure. Method 400 may be stored as executable instructions in non-transitory memory 106, for example, on server 101 and may be executed by one or more hardware processors 104 of the logic subsystem 103.

Method 400 begins at 405. At 405, method 400 receives a search query. For example, a user of a user device, such as user device 121, may enter text or other user input to form a search query, for example via user input subsystem 129. The user device 121 then transmits the search query over a network 113 to the server 101. Method 400 thus receives the search query from a user device such as the user device 121. The search query comprises the text string submitted by the user, as an illustrative example. Furthermore, in some examples, the search query may further indicate location information and/or health care plan information for the user, for example as input by the user, captured in metadata with the submission of the search query, and/or stored in a user profile for the user, in order to further refine or limit the search. As mentioned hereinabove, the user may comprise a person searching for healthcare providers on their own behalf (e.g., a patient) or on behalf of another person (e.g., a care navigator, a parent or guardian of the patient, and so on).

At 410, method 400 extracts one or more keywords from the search query. For example, the text string of the search query may be tokenized to separate each term of the search query into keywords. At 415, method 400 identifies one or more desired areas of focus based on the one or more keywords and an area-of-focus taxonomy. For example, method 400 may search taxonomies, via the taxonomy module 220 as an example, to identify taxonomies that include one or more keywords of the search query. Taxonomies may be scored according to relevancy to the search query, for example such that a taxonomy containing a match for two or more keywords may be considered more relevant than a taxonomy containing a match for a single keyword in the search query. Further, the taxonomies may connect various terms in plain and technical language to improve the search. For example, a search for “c section” may be broken into the keywords “c” and “section,” and a taxonomy for cesarean delivery may include both keywords. Consequently, method 400 may identify “cesarean delivery” as a desired area of focus for the search query.

At 420, method 400 searches a provider database based on the desired area(s) of focus. The results may be retrieved from a provider database, such as provider database 245, as a non-limiting example. Further, the search may be limited, for example, by the location information and/or the health care plan information associated with the search query. For example, the results retrieved from the provider database 245 may be limited to providers within the desired geographical region and/or who accept a specified insurance plan.

At 425, method 400 outputs a list of at least one result sorted by relevancy to the search query, the at least one result including provider information for at least one provider. The list may be displayed in a graphical user interface or otherwise in a user interface to a user. To that end, method 400 may output the list of the at least one result to the user device 121 for display to the user via the display subsystem 127, as an illustrative example. Further, the results of the search performed at 420 may include a plurality of providers associated with the desired area of focus identified at 415. The plurality of providers may be sorted according to the degree of relevancy to the desired area of focus. For example, a provider who outperforms other providers with regard to a certain procedure related to the area of focus may be listed higher than the other providers in the list. If two or more desired areas of focus are identified in the search query, then the list may be sorted such that providers who specialize in the two or more desired areas of focus are weighted higher and displayed more prominently relative to providers who only specialize in one of the two or more desired areas of focus. An example graphical user interface for displaying such a list of search results based on identified areas of focus is described further herein with regard to FIGS. 11A-11B. After outputting the search results, method 400 then returns.

FIG. 5 shows a high-level flow chart illustrating an example method 500 for processing healthcare claims to identify areas of focus for healthcare providers according to an embodiment. In particular, method 500 relates to evaluating claims for each encounter to determine areas of focus associated with providers, and evaluating the associations identified for each provider to identify areas of focus associated with healthcare providers. Method 500 may thus comprise a subroutine of the method 300 as described hereinabove. Method 500 is described with reference to the systems and components of FIGS. 1 and 2, though it should be appreciated that the method may be implemented with other systems and components without departing from the scope of the present disclosure. Method 500 may be stored as executable instructions in non-transitory memory 106, for example, on server 101 and may be executed by one or more hardware processors 104 of the logic subsystem 103.

Method 500 begins at 505. At 505, method 500 groups all claims associated with a same encounter. That is, for the raw medical claims retrieved from the claims database at 305, method 500 groups the claims based on encounter. An encounter comprises a specific care event for a patient, and thus may include all claims related to the specific care event. Numerous services can be performed within a medical event. For example, for an ACL repair surgery, within just the surgical event or encounter there may be claims for a surgeon procedure, anesthesiologist, assistant surgeon, facility fees, recovery rooms, medications, and so on. In some cases, multiple procedures are all performed in the same event. Thus, method 500 groups claims according to encounters.

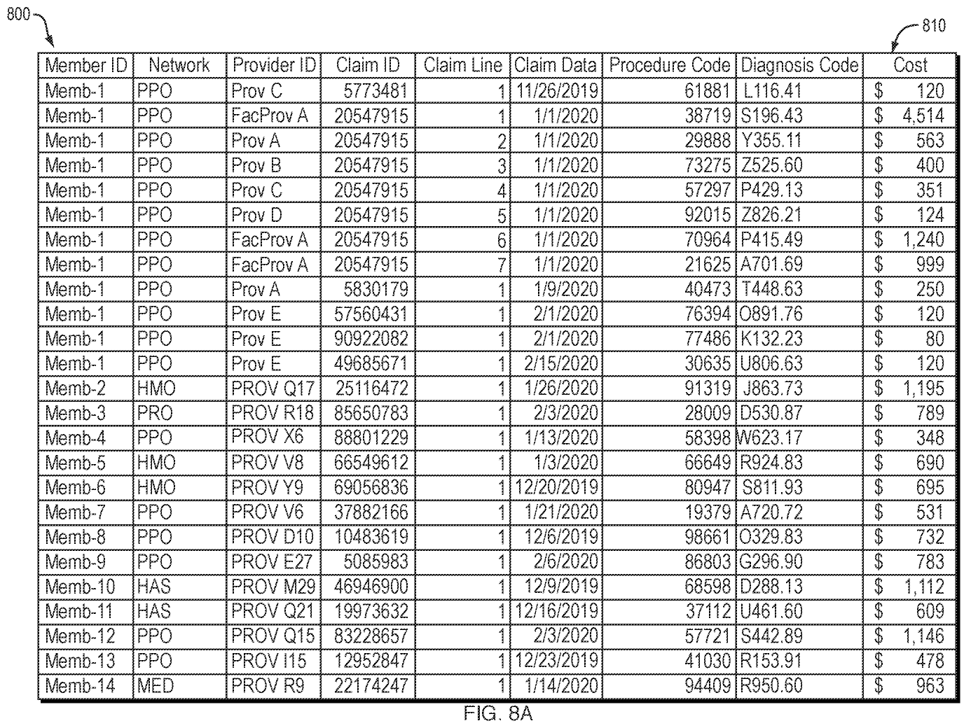

After grouping the claims into encounters, at 510, method 500 evaluates the claims for each encounter to determine one or more area(s) of focus and patient attributes associated with providers of the encounter. For example, method 500 evaluates the claims in each group to determine the primary service being performed in the encounter and the responsible provider, for example by evaluating the procedure, diagnosis, and modifier codes across the encounter. Thus, within the encounter grouping process, method 500 identifies the primary provider responsible for the encounter or event as well as the event itself. While the claim does not necessarily include information indicating a lead or primary doctor, method 500 identifies the primary provider based on other attributes. Further, as multiple services may occur within a single encounter, method 500 also identifies the primary service being provided such as ACL repair (rather than, say, the provision of anesthetics). Method 500 may identify the area of focus related to the encounter based on the primary service being provided. Further, method 500 may identify patient attributes of the patient for whom the service is being provided. The providers associated with the encounter may be associated with the area of focus and the patient attributes identified, for example by incrementing a counter for the area of service and the provider. As the claims are processed for a plurality of encounters, patterns may thus emerge as such counters are incremented. An example method for evaluating the claims for each encounter is described further herein with regard to FIG. 6.

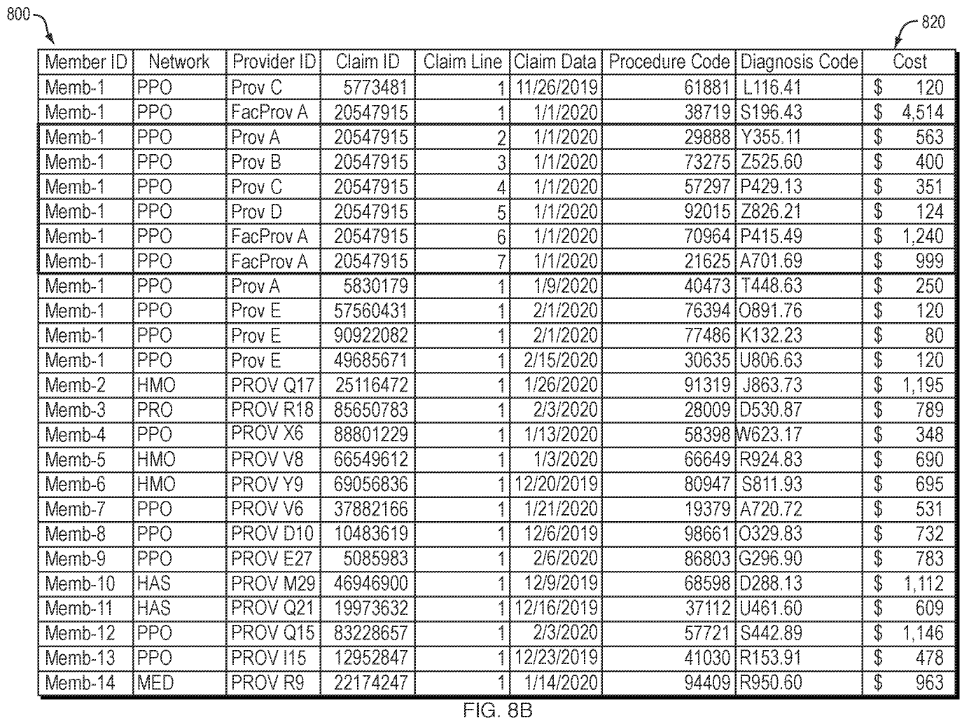

At 515, method 500 evaluates the associations for each provider to identify one or more areas of focus for each provider. For example, method 500 may determine whether a provider consistently provides services within a given area of focus for a plurality of encounters, and the number of services provided within a given area of focus may be considered relative to the number of similar services provided by similar providers. In this way, a provider may not be considered to specialize within an area of focus if the provider performs a substantially small number of services within the area of focus relative to other services provided by the provider, as well as relative to the number of services within the area of focus provided by other providers. An example method for evaluating the associations for each provider is described further herein with regard to FIG. 7.

At 520, method 500 updates the provider database with the area(s) of focus identified for each provider. For example, provider information for a given healthcare provider may be updated in the provider database 245 to indicate that the provider is a relatively high-performer within an area of focus (e.g., relative to other providers within the area of focus), or simply that the bulk of services provided by the provider are within the area of focus. The provider database may similarly be updated to indicate that a provider provides services to a relatively high number of patients with a given patient attribute. In this way, a search for healthcare providers as described herein, when performed within the provider database including detailed area of focus information, may provide more accurate and up-to-date results based on actual claim data rather than self-reported results from an expensive, manually-conducted survey. Method 500 then returns.

FIG. 6 shows a high-level flow chart illustrating an example method 600 for evaluating claims for an encounter to determine areas of focus and patient attributes associated with providers according to an embodiment. In particular, method 600 comprises an iterative subroutine of the method 500. Method 600 is described with reference to the systems and components of FIGS. 1 and 2, though it should be appreciated that the method may be implemented with other systems and components without departing from the scope of the present disclosure. Method 600 may be stored as executable instructions in non-transitory memory 106, for example, on server 101 and may be executed by one or more hardware processors 104 of the logic sub system 103.

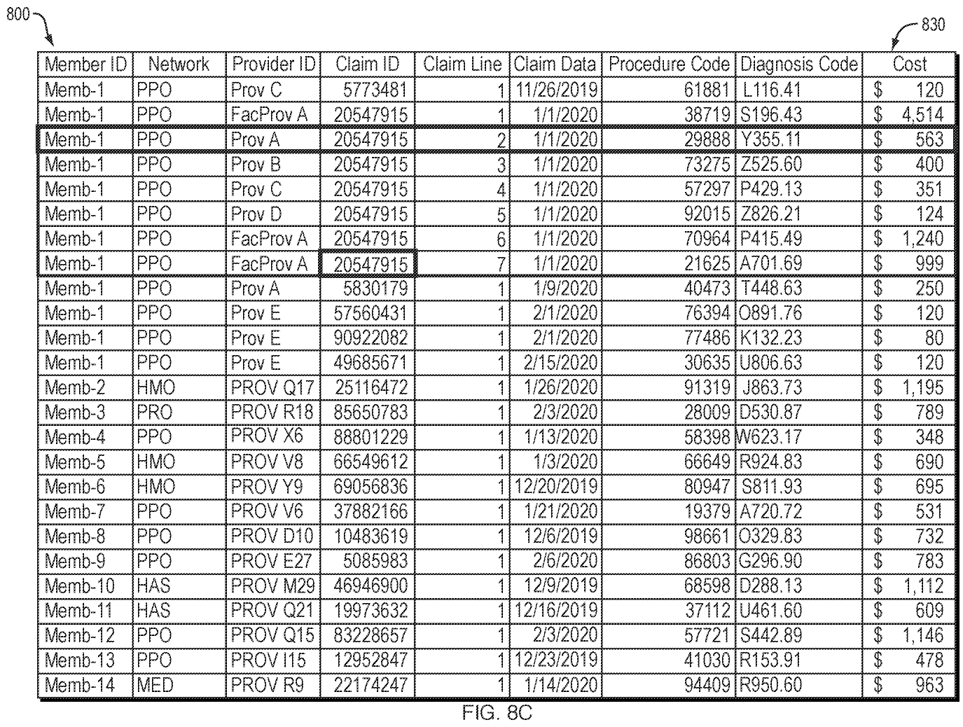

Method 600 begins at 605. At 605, method 600 iteratively processes or evaluates claims for each encounter until all encounters in the raw medical claims dataset are evaluated. As an illustrative and non-limiting example, FIGS. 8A-8C show a set of tables 800 of example claims illustrating a method for evaluating claims to identify areas of focus of providers. FIG. 8A includes a plurality of raw medical claims for a plurality of patients (i.e., members) and a plurality of encounters. FIG. 8B illustrates the grouping of a subset of claims in the raw medical claims that correspond to a particular healthcare encounter. For a given encounter, at 610, method 600 loads claims associated with the encounter. For example, method 600 may load the highlighted claims in FIG. 8B for evaluation.

At 615, method 600 determines a primary provider for the encounter based on the grouped claims. Method 600 may determine the primary provider for example, by comparing the procedure and/or diagnosis codes of the claims to a corresponding taxonomy. The taxonomy may indicate that a certain procedure, when grouped with other procedures, is the primary procedure. Method 600 may thus identify the primary procedure or service in the grouped claims, and identify the provider responsible for the primary procedure as indicated in the claims. FIG. 8C illustrates the primary provider for the primary service by highlighting the claim in red. Thus, at 620, method 600 also determines one or more areas of focus for the encounter based on procedure codes and/or diagnosis codes of the claims. Method 600 may further apply human-readable labels to the services, for example as designated by the corresponding taxonomy.

At 625, method 600 associates the one or more areas of focus with at least the primary provider. For example, method 600 may associate the one or more areas of focus determined at 620 with the primary provider determined at 615. Method 600 may further associate the one or more areas of focus with other providers identified by the claims. However, method 600 may down-weight the areas of focus for other providers relative to the primary provider. For example, the association of the one or more areas of focus with the providers may comprise a numerical value or score for the encounter. The score for the primary provider may be higher than the score for secondary or tertiary providers. After summing the scores for each provider and all encounters, the resulting summed scores for each area of focus may then be used to determine whether the provider specializes in the area of focus.

At 630, method 600 determines patient attributes for the patient of the encounter. For example, all claims associated with the patient, including claims outside the encounter, may be evaluated to identify one or more patient attributes for the patient. Such patient attributes may be stored in a patient attribute database 111, for example, and so method 600 may determine the patient attributes for the patient from the patient attribute database. In this way, attributes of the patient that may not be evident given the claims of the encounter or other patient information typically available may be obtained.

At 635, method 600 associates the patient attributes with at least the primary provider. For example, a patient attribute score for each patient attribute identified may be assigned to each provider, with a higher score being assigned to or associated with the primary provider. In this way, when evaluating all encounters for a given provider, the patient attribute scores may be summed in order to determine whether the provider specializes in providing care for patients with a given patient attribute. Such a score may indicate a cultural competency of the provider, for example.

At 640, method 600 updates the provider database, such as the provider database 245, with the associations. For example, method 600 may add the associations, which may comprise numerical scores for each area of focus and/or patient attribute, to the provider database for the provider.

Method 600 evaluates the claims for each encounter as described above until all encounters of the claims dataset are processed. Method 600 then returns.

FIG. 7 shows a high-level flow chart illustrating an example method for evaluating associations for providers to determine areas of focus for the providers according to an embodiment. In particular, method 700 comprises an iterative subroutine of the method 500. Method 700 is described with reference to the systems and components of FIGS. 1 and 2, though it should be appreciated that the method may be implemented with other systems and components without departing from the scope of the present disclosure. Method 700 may be stored as executable instructions in non-transitory memory 106, for example, on server 101 and may be executed by one or more hardware processors 104 of the logic subsystem 103.

Method 700 begins at 705. At 705, method 700 iteratively evaluates the associations for each healthcare provider associated with claims in the raw medical claims until the associations for all providers are evaluated. The associations are identified, as described hereinabove with regard to FIG. 6, by evaluating healthcare claims for each encounter as well as for each patient.

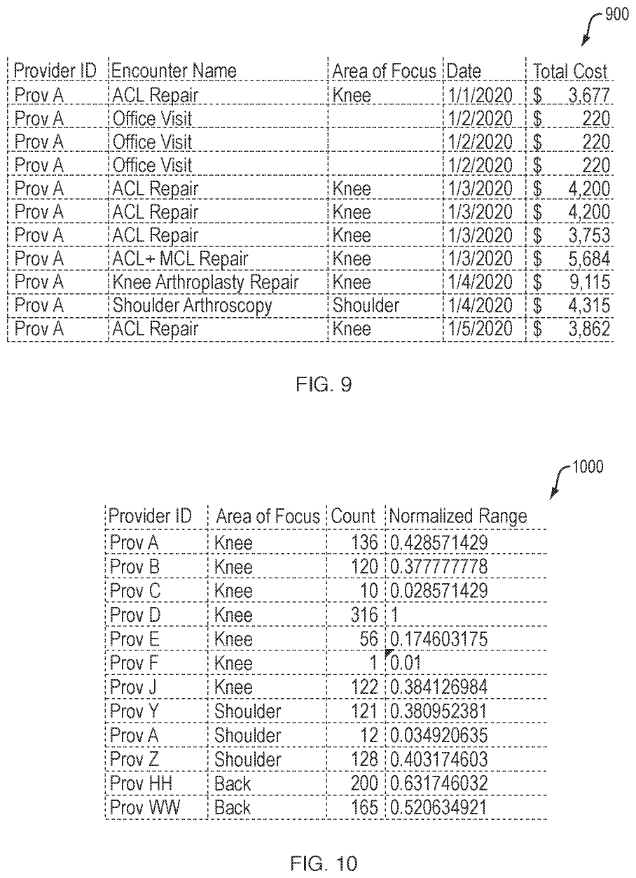

Thus, for a given provider, at 710, method 700 evaluates associations for the provider. For example, method 700 evaluates the number of associations of a provider with a given area of focus and/or patient attribute. Each association of a provider with a given area of focus and/or patient attribute may be weighted, for example depending on the primacy of the provider in the provision of a service within the area of focus. As an illustrative and non-limiting example, FIG. 9 shows a table 900 illustrating example areas of focus associated with a provider. The table 900 in particular illustrates example encounters and areas of focus identified in raw claims data and associated with the provider, as described hereinabove with regard to FIG. 6. As depicted, a provider (Prov A) mostly provides services within the area of focus “Knee” though also provides services within the area of focus “Shoulder.”

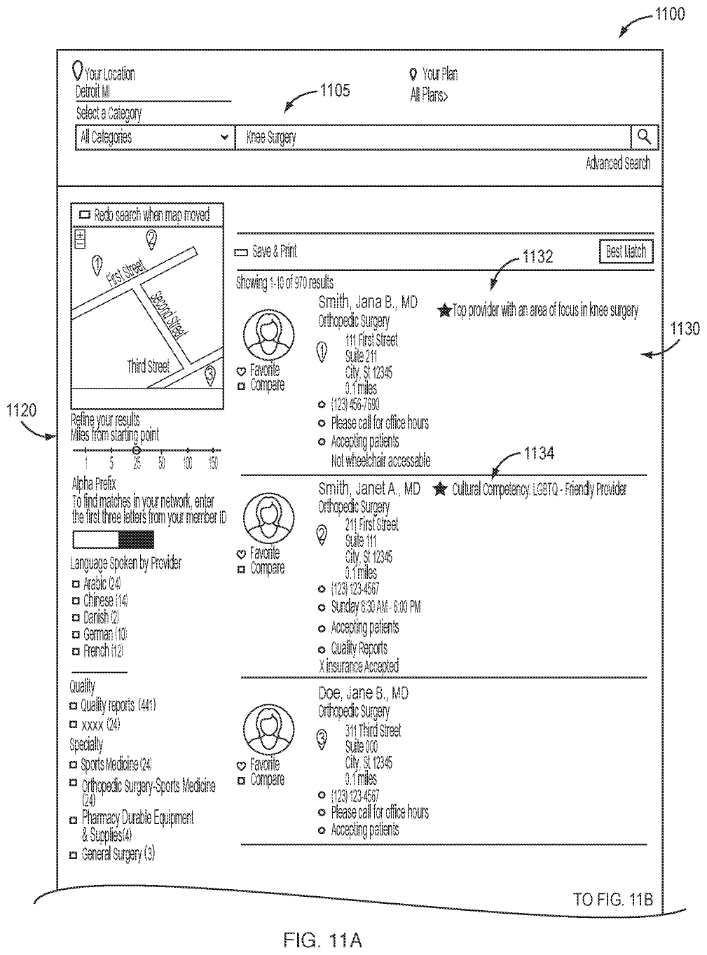

Referring again to FIG. 7, at 715, method 700 determines whether one or more areas of focus are above a baseline rate. The baseline rate may comprise a relative rate determined based on associations for a plurality of providers within a given area of focus. For example, the numerical scores indicating the associations for all providers within an area of focus may be normalized, and the baseline rate may be determined based on the normalized numerical scores. The baseline rate may be manually selected or automatically determined such that providers with a number of associations or a numerical score above the baseline rate may be considered to specialize in the area of focus. As an illustrative and non-limiting example, FIG. 10 shows a table illustrating example data for determining areas of focus for providers. The associations for the provider (Prov A) discussed hereinabove with regard to FIG. 9 are summed into a count and a normalized range (relative to other providers) is also calculated, as depicted, while associations for other providers are also calculated. Thus, when determining areas of focus for the provider (Prov A), the total number of associations for each area of focus may be considered for the provider as well as relative to other providers. As depicted, the provider (Prov A) provides services within the area of focus “Knee” as well as “Shoulder” but not within the area of focus “Back.” Further, the provider (Prov A) provides a substantial number of services within the area of focus “Knee” relative to other providers, while providing fewer services within the area of focus “Shoulder” relative to the number of services provided for “Knee” as well as other providers working within the “Shoulder” area of focus. The baseline rate may comprise a numerical threshold for the normalized range. A separate baseline rate may be determined or provided for each area of focus.

If at least one area of focus is above the baseline rate (“YES”), method 700 continues to 720. At 720, method 700 assigns the at least one area of focus above the baseline rate to the provider. The provider may thus be designated as specializing in the area of focus. Further, if the associations for the provider indicates that the provider is in a higher percentile of providers for providing the service, method 700 may assign an additional designation indicating the relatively higher performance of the provider within the area of focus. Method 700 then proceeds to 725.

Referring again to 715, if no areas of focus are above the baseline rate (“NO”), method 700 proceeds directly to 725. In either case, at 725, method 700 determines whether one or more patient attributes are above an attribute baseline rate. The attribute baseline rate comprises a threshold rate selected or automatically determined such that if a number of associations of the provider with the patient attribute is above the attribute baseline rate, then the provider may be considered to specialize in providing care for patients with the given patient attribute. If at least one patient attribute is above a baseline rate (“YES”), method 700 continues to 730. At 730, method 700 assigns the at least one patient attribute above the attribute baseline rate to the provider. Method 700 may further assign a designation to the provider if the rate of care for the given patient attribute is in a highest percentile, such that the provider is a particularly notable provider of care for patients with the patient attribute. Method 700 then proceeds to 735.

Referring again to 725, if no patient attributes are above the attribute baseline rate (“NO”), method 700 proceeds to 735. At 735, method 700 updates the provider database with the assignments assigned at 720 and/or 730. If no assignments are assigned at 720 or 730, then method 700 may still update the provider database 245 with the results of the evaluation of the associations for the provider. For example, with regard to the example data depicted in FIG. 10, the provider (Prov A) may be associated with the “Knee” and “Shoulder” areas of focus, due to the relatively higher provision of services within the “Knee” area of focus such that the normalized range is above a baseline rate, the provider may only be distinctly associated with the “Knee” area of focus. However, the provider database may still be updated to indicate the “Shoulder” area of focus such that the provider may be included in search results for both areas of focus, though the provider may be considered less relevant than the providers Prov Y and Prov Z within the “Shoulder” area of focus. Once method 700 evaluates the associations for each provider, method 700 returns.

FIGS. 11A-11B show a diagram illustrating an example graphical user interface 1100 for displaying search results based on areas of focus according to an embodiment. The graphical user interface 1100 may be displayed to a user of a user device 121 via a display subsystem 127, as an illustrative and non-limiting example.

The graphical user interface 1100 includes a search query input 1105. As depicted, a user has input “knee surgery” to the search query input 1105. The systems and methods described herein may identify “Orthopedic Surgery” as a top context for the search based on a comparison of the search terms to a structured dictionary, and furthermore may identify a “Knee” area of focus within the orthopedic surgery specialization. As such, the search results 1130 comprise providers who specialize in orthopedic surgery and whose area of focus is “Knee,” as depicted. The search results 1130 are sorted such that providers with a higher association with the “Knee” area of focus are displayed higher in the results 1130. Further, the graphical user interface 1100 may include an indicator 1132 positioned adjacent to provider information for a provider in a highest percentile within the area of focus. In this way, a user may discern that the provider is a high or top performer within the area of focus relative to other providers in the search results 1130. The graphical user interface 1100 may further include an indicator 1134 to indicate a cultural competency for a given provider in the results 1130, which indicates that the provider treats an unusually high percentage of patients with a given patient attribute. In this way, users may identify providers with certain experience who may be able to provide improved care given their unique needs.

Further, the results may include providers who include at least one association with the desired area of focus (“Knee”) despite specializing in another area of focus. The graphical user interface 1100 may indicate such providers via an indicator 1138 displayed in the graphical user interface 110 that indicates the area of focus for the provider.

Additionally, the interface 1100 includes a plurality of search categories 1120, unrelated or related to the search contexts, that allow the user to refine the results, for example based on languages spoken by the provider, quality, specialty, areas of focus, provider type, gender, location services, and distance from the user. Selections in the plurality of search categories 1120 may be submitted via the interface 1100 along with the search query input 1105 to limit the search accordingly.

It will be appreciated that the configurations and routines disclosed herein are exemplary in nature, and that these specific embodiments are not to be considered in a limiting sense, because numerous variations are possible. The subject matter of the present disclosure includes all novel and non-obvious combinations and sub-combinations of the various systems and configurations, and other features, functions, and/or properties disclosed herein.

The following claims particularly point out certain combinations and sub-combinations regarded as novel and nonobvious. Such claims should be understood to include incorporation of one or more such elements, neither requiring nor excluding two or more such elements. Other combinations and sub-combinations of the disclosed features, functions, elements, and/or properties may be claimed through amendment of the present claims or through presentation of new claims in this or a related application.

Such claims, whether broader, narrower, equal, or different in scope to the original claims, are also regarded as included within the subject matter of the present disclosure.

1. A computer-implemented method, the method performed by one or more hardware processors, comprising:
processing raw medical claims to identify an area of focus for a provider;
associating the provider with the area of focus;
receiving, from a user via a user device, a search query indicating a desired area of focus; and
outputting, to the user device for display to the user, provider information of the provider if the desired area of focus comprises the area of focus.

2. The method of claim 1, wherein processing the raw medical claims to identify the area of focus for the provider comprises:
grouping claims of the raw medical claims into a plurality of groups, each group associated with an encounter;
for each grouping of claims, evaluating the claims associated with the encounter to determine an association of at least one area of focus and the provider; and
evaluating the associations for the provider determined from each grouping of claims to identify the area of focus.

3. The method of claim 2, wherein evaluating the claims associated with the encounter to determine the association of the at least one area of focus and the provider comprises determining the provider as a primary provider for the encounter, determining the at least one area of focus for the encounter based on one or more of a procedure code and/or a diagnosis code, and associating the at least one area of focus with the provider.

4. The method of claim 3, further comprising determining the at least one area of focus for the encounter based on one or more of the procedure code and/or the diagnosis code according to a taxonomy for the at least one area of focus, wherein the taxonomy links the at least one area of focus to one or more of the procedure code and/or the diagnosis code.

5. The method of claim 3, wherein evaluating the associations for the provider determined from each grouping of claims to identify the area of focus comprises determining that a number of associations of the provider with the area of focus is above a baseline rate.

6. The method of claim 5, further comprising determining the baseline rate relative to a number of associations of the area of focus with other providers.

7. The method of claim 1, further comprising processing claims of the raw medical claims for a patient to identify a patient attribute, assigning the patient attribute to the provider if the provider treats a number of patients associated with the patient attribute above an attribute baseline rate, and displaying, in the provider information during display to the user, an indication that the provider specializes in the patient attribute if the patient attribute is assigned to the provider.

8. A system, comprising:
a hardware processor; and
a non-transitory memory device coupled to the hardware processor and storing executable instructions that when executed cause the hardware processor to:
process raw medical claims to identify an area of focus for a provider;
associate the provider with the area of focus;
receive, from a user via a user device, a search query indicating a desired area of focus; and
output, to the user device for display to the user, provider information of the provider if the desired area of focus comprises the area of focus.

9. The system of claim 8, wherein processing the raw medical claims to identify the area of focus for the provider comprises:
grouping claims of the raw medical claims into a plurality of groups, each group associated with an encounter;
for each grouping of claims, evaluating the claims associated with the encounter to determine an association of at least one area of focus and the provider; and
evaluating the associations for the provider determined from each grouping of claims to identify the area of focus.

10. The system of claim 9, wherein evaluating the claims associated with the encounter to determine the association of the at least one area of focus and the provider comprises determining the provider as a primary provider for the encounter, determining the at least one area of focus for the encounter based on one or more of a procedure code and/or a diagnosis code, and associating the at least one area of focus with the provider.

11. The system of claim 10, wherein the executable instructions when executed further cause the hardware processor to determine the at least one area of focus for the encounter based on one or more of the procedure code and/or the diagnosis code according to a taxonomy for the at least one area of focus, wherein the taxonomy links the at least one area of focus to one or more of the procedure code and/or the diagnosis code.

12. The system of claim 10, wherein evaluating the associations for the provider determined from each grouping of claims to identify the area of focus comprises determining that a number of associations of the provider with the area of focus is above a baseline rate.

13. The system of claim 12, wherein the executable instructions when executed further cause the hardware processor to determine the baseline rate relative to a number of associations of the area of focus with other providers.

14. The system of claim 8, wherein the executable instructions when executed further cause the hardware processor to process claims of the raw medical claims for a patient to identify a patient attribute, assign the patient attribute to the provider if the provider treats a number of patients associated with the patient attribute above an attribute baseline rate, and display, in the provider information during display to the user, an indication that the provider specializes in the patient attribute if the patient attribute is assigned to the provider.

15. The system of claim 13, wherein the executable instructions when executed further cause the hardware processor to transmit a search context of the one or more search contexts not used for the search to the user device for display as an alternate search suggestion.

16. The system of claim 8, wherein the non-transitory memory device further stores a score module configured to calculate the score for each of the one or more search contexts.

17. A computer-readable storage medium storing a program of instructions executable by a machine to perform a method, the method comprising:
processing raw medical claims to identify an area of focus for a provider;
associating the provider with the area of focus;
processing the raw medical claims to identify a patient attribute of patients treated by the provider;
associating the provider with the patient attribute;
receiving, from a user via a user device, a search query indicating a desired area of focus; and
outputting, to the user device for display to the user, provider information of the provider if the desired area of focus comprises the area of focus, the provider information further indicating that the provider specializes in serving patients with the patient attribute.

18. The computer-readable storage medium of claim 17, wherein processing the raw medical claims to identify the area of focus for the provider comprises:
grouping claims of the raw medical claims into a plurality of groups, each group associated with an encounter;
for each grouping of claims, evaluating the claims associated with the encounter to determine an association of at least one area of focus and the provider; and
evaluating the associations for the provider determined from each grouping of claims to identify the area of focus.

19. The computer-readable storage medium of claim 17, further comprising processing the raw medical claims to identify the area of focus for a provider based on a taxonomy that connects codes of the raw medical claims to the area of focus.

20. The computer-readable storage medium of claim 19, further comprising generating the taxonomy by applying a machine learning clustering algorithm to the raw medical claims to cluster the raw medical claims and applying a label to each cluster.
